(19) United States
(12) Patent Application Publication
(10) Pub. No.: US 2022/0157440 A1
(43) Pub. Date: May 19, 2022

(54) System and Method for Credentialing and Scheduling On-Call Provider Services for Hazard Events

(71) Applicant: iDimension Applications, LLC, Palmer, AK (US)

(72) Inventor: Samuel Korsmo, Palmer, AK (US)

(21) Appl. No.: 17/402,144

(22) Filed: Aug. 13, 2021

Related U.S. Application Data
(60) Provisional application No. 63/115,525, filed on Nov. 18, 2020.

(51) Int. Cl. G16H 40/20; G16H 40/40; G06Q 10/06

(52) U.S. Cl. CPC G16H 40/20; G06Q 10/06311; G16H 40/40

(57) ABSTRACT

The invention provides a system and method of organizing medical personnel and other providers during a hazard event. A system links information about medical personnel and providers with national, state, and local medical facilities and entities. A method provides medical personnel and providers a means of enlisting in various hazard response efforts and affords administrators of medical facilities the ability to authorize and schedule the medical personnel and providers in an efficient manner.

### TECHNICAL FIELD

The present invention is directed to the management of healthcare provider credentialing and scheduling in response to a hazard event.

### BACKGROUND

With the passage of the Emergency Medical Treatment and Labor Act of 1986 (“EMTALA”), hospital facilities were obligated to maintain on-call availability schedules. The act requires hospital facilities to triage patients arriving at the facility, regardless of their ability to pay. This expanded demand on hospitals necessitated the placement of healthcare providers on availability schedules to prepare for potential excess of patients.

Continuing to the present, on-call schedules for healthcare providers are maintained for every hospital in the United States. In order to serve at a hospital, each provider must be licensed in a state and have authority (“Credentials”) to work at that particular facility.

EMTALA schedules may be referred to as “EMTALA On-Call Schedule(s)”. These generally provide twenty-four-hour periods of time where a provider is available to report to the hospital should a need arise.

Inevitably, from time to time, increased needs beyond the EMTALA On-Call Schedules occur during hazard events. Examples could be natural or manmade, and include hurricanes, floods, earthquakes, terrorism, chemical spills, etc. Additional methods of sourcing and managing healthcare providers are needed.

The U.S. Department of Health and Human Services, Office of the Assistant Secretary for Preparedness and Response has created a national list of healthcare providers who have registered to assist in anticipated disaster events. This list is called the Emergency System for Advance Registration of Volunteer Health Professionals (“ESAR-VHP”). Any provider with a National Provider Identifier (“NPI”) may volunteer for placement on the ESAR-VHP list.

State governments have access to the national ESAR-VHP list and use it to create their own lists of volunteers. The ESAR-VHP list provides general information about the provider, including the NPI, license, and Credentials.

When a disaster strikes, the local and state authorities are often overwhelmed with healthcare needs, but inadequately prepared to source and allocate providers. The current ESAR-VHP and EMTALA On-Call Schedule systems are incapable of performing this task. The current ESAR-VHP lists do not contain scheduling information for the volunteer providers, and the EMTALA On-Call Schedules are designed for a separate, incompatible function. EMTALA On-Call Schedules provide routine overflow outside of the current hazard, meaning those providers are otherwise already allocated for standard emergency medical treatment and are thus not available for the immediate hazard response effort.

There are three separate systems for EMTALA On-Call Schedules, ESAR-VHP, and for each hospital's credentialing. These systems were designed without the others in mind. When disaster strikes, emergency response administrators are often unprepared, and unorganized. They find it difficult to source licensed and credentialed providers for the immediate and ongoing needs of dealing with the crisis.

The Centers for Medicare & Medicaid Services (“CMS”) has acknowledged a need for general preparedness in view of all hazards and issued a Rule for “Emergency Preparedness Requirements for Medicare and Medicaid Participating Providers and Suppliers” on Sep. 16, 2016. Among other requirements, the CMS requires efficient communications for organizing providers. The Rule states, “During an emergency, it is critical that hospitals, and all providers/suppliers, have a system to contact appropriate staff, patients' treating physicians, and other necessary persons in a timely manner to ensure continuation of patient care functions throughout the facilities and to ensure that these functions are carried out in a safe and effective manner.”

There is a need for an improved hazard response system, that can find licensed providers, facilitate rapid credentialing for affected hospital facilities, and schedule the providers swiftly and efficiently with forethought.

### SUMMARY

### Glossary of Terms

The following is a list of pertinent terms of art for the disclosed invention. Terms are capitalized throughout this document and may be written in the singular or plural as needed.

Administrator—the manager of a Facility capable of scheduling and granting Credentials to Providers.

All-Hazard Event—any emergency requiring additional personnel.

CMS—Centers for Medicare & Medicaid Services, a division of Health and Human Services.

Credentials—authorization to work at a specific Facility and perform specific procedures.

DEA License—a federal license granted by the Drug Enforcement Administration to administer and prescribe controlled substances.

EMTALA—Emergency Medical Treatment and Labor Act of 1986, mandated the offering of emergency medical treatment to all patients.

EMTALA On-Call Schedule—twenty-four-hour periods outside of a Provider's Practice Schedule or Group Schedule, where a Facility may request the Provider to assist during an emergency.

ESAR-VHP—Emergency System for Advance Registration of Volunteer Professionals, a list maintained by the U.S. Department of Health and Human Services of providers who have volunteered to assist in a hazard event.

Facility—an organization providing healthcare or emergency services. This includes hospitals and incidence command centers, operated by government, non-profits, or private companies, whether permanent or temporary.

Group—a collection of Providers with similar or collaborative specialties operating under a joint schedule.

Group Schedule—a Provider's regularly planned working hours within a Group.

Hazard Event—an emergency of a particular type with the propensity for human casualties, requiring additional medical and/or emergency response personnel.

Hazard On-Call Schedule—allotted periods of time where a Facility may request a Provider to assist during a Hazard Event.

Independent Provider—a Provider that is not affiliated with a Group.

NPI—National Provider Identifier, a unique number for health care providers maintained by the Centers for Medicare & Medicaid Services.

Practice Schedule—an Independent Provider's regularly planned working hours.

Provider—includes doctors and nurses with NPIs, as well as other healthcare and emergency personnel, such as emergency medical technicians, medical assistants, therapists, technicians, Administrators, first responders, and any other support personnel.

Provider Table—a database table of information regarding a particular Provider, including their specialty, licenses, NPI, location, and contact details.

Provisional Credentials—temporary authorization to work at a specific Facility and perform specific procedures.

State Medical License—a license granted by a state or territory medical board, permitting the licensee to provide healthcare services in that state or territory.

User—A Provider, Administrator, or other person with access to the invention's system.

Vacation Schedule—a Provider's planned time away from their Practice Schedule or Group Schedule.

### SUMMARY OF THE INVENTION

The present invention provides a system and method for preparing and managing healthcare Providers responding to a Hazard Event. The invention organizes healthcare Providers according to their State Medical Licenses, Credentials, skills, and availability, in a way that affords hospital Facilities and emergency management Administrators to efficiently find and credential Providers, and staff those Providers to where they are most needed.

The system manages a variety of people with various skills required to respond to an emergency. In this system, emergency responders are generally called Providers, which include medically licensed doctors and nurses, but may also include other personnel without State Medical Licenses.

Providers enroll in the system to make themselves available for support during a Hazard Event. Providers may select particular types of hazards or may make themselves available for any type of hazard. Providers' locations and contact information are included. The system connects information from respective Facilities and government agencies to include State Medical Licenses, NPIs, DEA Licenses, and Credentials.

Additionally, the system includes Providers' current schedules, possibly including one or more of their EMTALA On-Call Schedules, Group Schedules, Practice Schedules, and Vacation Schedules.

Participating Facilities have access to the list of Providers, and whether that Provider has Credentials at the Facility. Each Facility has an Administrator with authority to permit medical personnel to serve there. Administrators also have access to the various schedules of each Provider.

When a Hazard Event occurs, one or more Facilities respond to the hazard. The Administrator at a responding Facility starts a process of meeting the heightened demand for medical and support personnel. The Administrator begins by searching through the system to find appropriate Providers for a new Hazard On-Call Schedule. If a Provider is available and has Credentials at that Facility, the Administrator may add them to the new Hazard On-Call Schedule. If a Provider does not have Credentials, the Administrator may grant the Provider Credentials for a limited time and purpose, enabling the Provider to serve immediately.

The Administrator schedules the Providers according to skill, location, and availability, taking their other schedules into consideration. Administrators may adjust the Hazard On-Call Schedule by adding or removing Providers as needed, and eventually retire the schedule after the need is over.

### LIST OF FIGURES

FIG. 1 is a diagram showing system components of one embodiment of the invention, external data sources, a web server, and a User device, connected by the Internet.

FIG. 2 is database table relationship diagram showing the connection of various data sources into a Provider table.

FIG. 3 is an example Provider table showing general information about Providers enrolled in the system.

FIG. 4 is an example display of information about a single Provider.

FIG. 5 is an entity relationship diagram showing the various sources of information necessary to create a Hazard On-Call Schedule.

FIG. 6 is a database table relationship diagram showing the connection of various Provider schedules.

FIG. 7 is a flowchart demonstrating one embodiment of the method for creating and adding Providers to a Hazard On-Call Schedule.

FIG. 8 is an example display of a Hazard On-Call Schedule.

### DETAILED DESCRIPTION

The present invention provides a system and method for hazard response Administrators to organize healthcare Providers before and during a Hazard Event. The invention connects information from multiple sources and presents it in such a way that enables an Administrator to find available healthcare Providers, grant them Credentials if necessary, and schedule them.

Data Sources

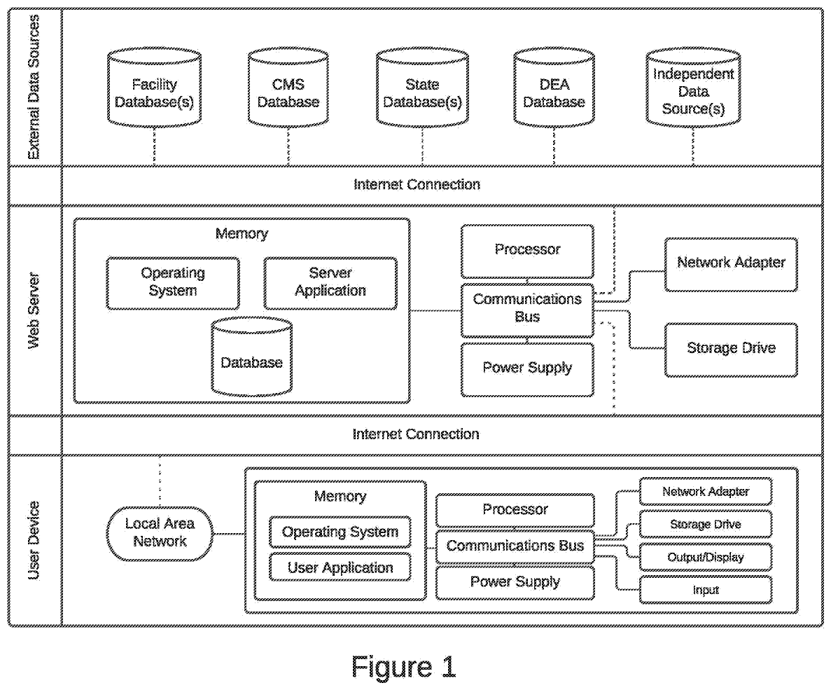

FIG. 1 is a diagram showing system components of one embodiment of the invention. The system includes links to external data sources, including Facility, CMS, state, DEA and Independent Data Sources, which all hold information about the Providers. The external data sources are connected to a web server which hosts the present invention's method of operation. Users connect to the web server and manage the system through local devices.

Facilities have their own systems with databases possessing information about their affiliated Providers. Facilities have biographical information about the Providers, including names, titles, phone numbers, specialties, fellowships, and Credentials. Facilities participating in the present invention's system supply this information for every Provider enrolled. Facilities also may possess EMTALA Schedules if the Provider participates in EMTALA, Group Schedules for any affiliated Provider associated with a Group operating at the Facility, and Vacation Schedules.

CMS is a federal agency that manages a national registry of unique identification numbers, NPIs, for health care providers. The NPI is used for any service under Medicare and is a requirement for all standard HIPAA (Health Insurance Portability and Accountability Act of 1996) compliant transactions. The present invention's system links to a database at CMS to verify the NPI for applicable enrolled Providers. Many Providers in the disclosed system, including physicians, nurse practitioners, and physical therapists have NPI's, but the definition of Provider herein also includes many emergency responders without NPI's.

In the United States, every state and territory has a medical board responsible for licensing physicians and other healthcare providers to practice medicine. Those medical boards grant and maintain physicians' licensing statuses. The present invention's system links to state databases to verify the status of each applicable Provider's State Medical License.

The Drug Enforcement Administration (“DEA”) regulates the production and distribution of controlled substances in the United States. The DEA maintains licensing information for those authorized to handle and administer controlled substances, including medicine essential to healthcare. The present invention's system links to DEA databases to verify the status of each applicable Provider's DEA License(s).

Some Providers enrolled in the system are Independent Providers and are not affiliated with a Facility or Group. For these Providers, the present invention's system may connect to an Independent Data Source containing biographical information about the Independent Provider, including name, title, phone number, specialty, fellowships, Credentials, and geolocation. The Independent Data Source may also maintain a Practice Schedule and Vacation Schedule for the Provider.

It is envisioned that future embodiments of the invention will possess additional connections to external data sources providing information about the Providers, Facilities, local government resources, weather conditions, and other information relevant to responding to Hazard Events.

The system is comprised of a web server with a memory, processor, and communications interface, hosting a web application. One embodiment of the invention uses Amazon Web Services, and a Windows Service written in C#.net. The web server also hosts a database with a Microsoft SQL server hosting the accumulated data and stored procedures. The web application exchanges information through a communications interface with external databases. In one embodiment, data is pulled from external data sources through the Internet by a Windows Service. One embodiment of the invention utilizes the Lightning Bolt proprietary API, and scheduling data is managed by Qgenda proprietary software.

### Hazard Events

Natural and manmade hazards may be divided into various categories of Hazard Events and prepared for accordingly. Potential Hazard Events include natural causes (e.g., tornadoes, severe storms, hurricanes, tropical storms, floods, wildfires, pandemics, earthquakes, droughts) and manmade causes (e.g., industrial accidents, mass shootings, general mass casualties, nuclear disasters, biological disasters, chemical disasters). These and other Hazard Events have a propensity for human casualties and necessitate the response of medical and other emergency personnel.

### Providers

Healthcare professionals and hazard response support personnel (“Providers”) include licensed doctors and nurse practitioners, as well as other non-licensed individuals. Example licensed Providers may include Surgeons, General Practitioners, Emergency Medicine Physicians, Anesthesiologists, Orthopedics Surgeons, Neurosurgeons, Cardiology Physicians, Cardiothoracic Surgeons, Radiologists, Infectious Disease Physicians, Ophthalmologists, and Nurse Practitioners. Example non-licensed individuals may include Physician Assistants, Chiropractors, Dentists, Dental Assistants, Therapists, Technicians, Nursing/Medical Assistants (Certified Nursing Assistants, Mid-wives, Radiological Technicians), Managers, Assistants, Financial Workers, Bookkeepers, Administrators, and Security Officers. Any person an Administrator deems helpful in response to a Hazard Event, may be included in the system as a Provider.

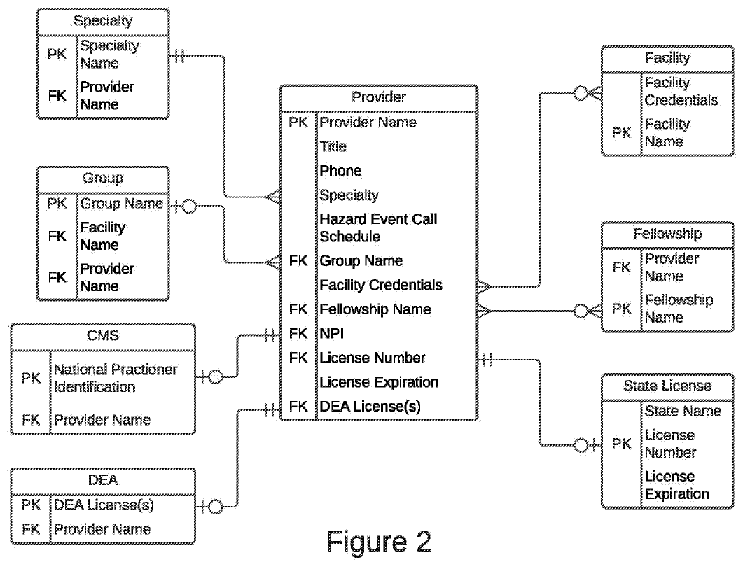

FIG. 2 is a database table relationship diagram. The center table, labeled “Provider” is the Provide Table. An Administrator with access to the system can view this information about Providers when making scheduling decisions. The Provider Table is created by linking information from the various data sources shown in FIG. 1. General Provider information, such as name, title, phone, and specialty may come from a participating Facility's Database, an Independent Data Source associated with an Independent Provider, or may be entered into the system by the Providers themselves.

Each Provider has one specialty, and the Provider Table links to a separate table organizing all Providers and their specialties. See the “Specialty” table in FIG. 2. For Providers associated with a Facility, a separate table organizes all Providers associated with that Facility and whether that Provider has Credentials at the Facility. See the “Facility” table. For Providers associated with a Group, a separate table organizes all Providers associated with that Group. See the “Group” table. Likewise, some Providers may be associated with a Fellowship, and thus are included in a separate “Fellowship” table.

Information regarding Credentials is supplied by Facilities' Databases. Information regarding Group membership, fellowship membership, and specialty may come from a Facility's Database or an Independent Data Source.

The “CMS” table is populated with information from the CMS Database, and supplies the NPI number associated with applicable Providers. The “DEA” table is populated with information from the DEA Database, and supplies information regarding the various DEA Licenses and privileges for applicable Providers. The “State License” table is populated with information from various U.S. State and Territory Databases, and supplies State Medical License information for applicable Providers.

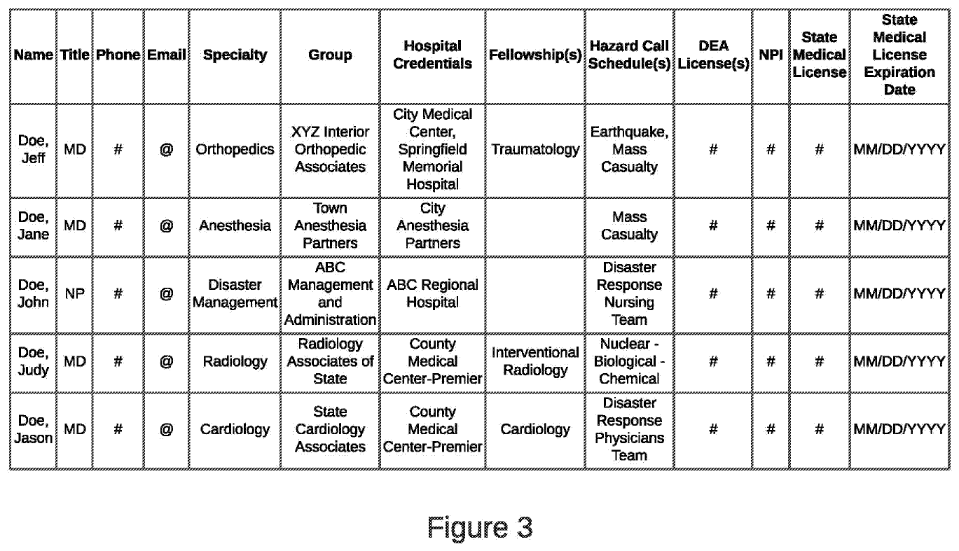

FIG. 3 is an example of some information from the Provider Table in a readable tabular format. When searching through the system to create a Hazard On-Call Schedule, an Administrator may review Providers in a list, and search for those most appropriate to respond to the current Hazard Event.

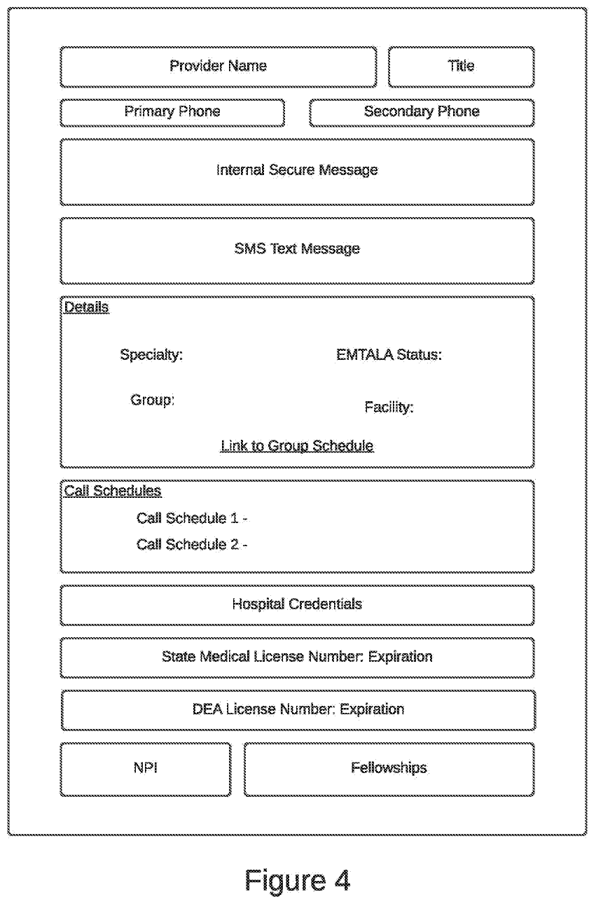

If a particular Provider looks appropriate to an Administrator, they can open a display for that single Provider. See FIG. 4. This display includes all the information necessary to assess the suitability of the Provider, as well as the Provider's various scheduling obligations.

The method of the present invention allows Providers to opt in. Prior to a Hazard Event, Providers may enroll in the system, providing information about themselves such that they are ready before they are needed. In some embodiments of the invention, the Provider may specify certain criteria for their involvement in a potential hazard response. Some limitations on participation may include proximity to the location, type of hazard, skillset, medical procedures, time and date, Facilities, etc. An Administrator may set up the system to afford selectivity on the part of Providers, that would increase the effectiveness of subsequent searches during a Hazard Event.

The web application exchanges information with Providers through a communications interface with Providers' devices and in some cases, their Independent Data Sources. Providers' devices are comprised of a memory, processor, communications interface, an input device, and an output display. A typical Provider device might be a personal computer, smartphone, or tablet. The web application displays information for the Providers to interface with the system. In one embodiment, the front end of the application is written in JavaScript, XML and CSS.

In one embodiment, the Windows Service regularly queries the web application's database, pulls data from the Independent Data Source, and sends scheduling information to the Providers. In some embodiments of the invention, the Windows Service sends daily emails or text messages to alert Providers of their scheduling status.

### Facilities

In one embodiment, the invention utilizes hospitals and incidence command centers, as the primary locus for hazard response. Typically, a hospital system provides the organization of treatment, but other embodiments of the invention may include government, non-profit, and other independent healthcare organizations. A Facility may refer to any healthcare treatment location, including a hospital or incidence command center, whether permanent or temporary.

Some Facilities (e.g., hospitals) are highly regulated institutions that require each Provider has Credentials to serve. Credentials are granted by an Administrator at the Facility, and last for a designated time, usually the life of the Provider's State Medical License. Credentials authorize the Provider to work at the Facility and dictate the privileges of performing various types of procedures.

Administrators may grant Credentials to new Providers if they meet certain criteria, for example, if the Provider can show a Medical State License and an NPI number. Administrators may also revoke Credentials, preventing the Provider from continued service at the Facility.

### Schedules

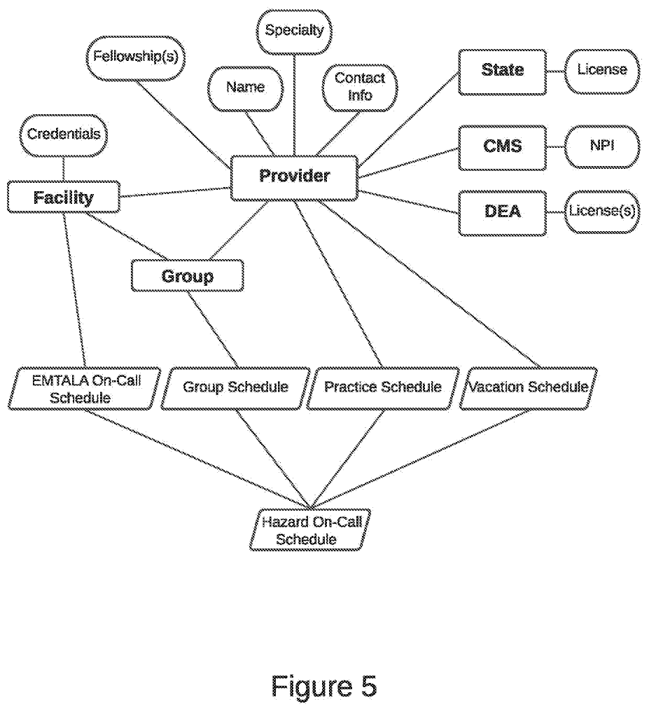

During a Hazard Event, an Administrator will create a Hazard On-Call Schedule for Providers to respond to the increased need. To create the Hazard On-Call Schedule, the Providers' existing other schedules may be considered. FIG. 5 is an entity relationship diagram showing the various types of information necessary to create a Hazard On-Call Schedule. A Provider may have an EMTALA On-Call Schedule, Group Schedule, Practice Schedule, and Vacation Schedule, all of which could affect their ability to join a new Hazard On-Call Schedule.

EMTALA On-Call Schedules are schedules created by Facilities to support emergency departments. For an individual Provider, they are generally for twenty-four-hour periods, a few days a month. If an emergency event at a Facility requires additional assistance, the Administrator may request the Provider on-call to come into the Facility. Some Providers are exempt from EMTALA requirements and do not participate in an EMTALA On-Call Schedule. EMTALA On-Call Schedules may overlap and superseded the other schedules listed below. Facilities provide information about EMTALA On-Call Schedules to the system of the present invention.

Group Schedules are schedules created by a Group of associated Providers. The Group may include similarly skilled or otherwise complimentarily skilled Providers. A Group may work entirely within one Facility or serve at multiple Facilities. The Group Schedule maintains the general working hours for the Providers in the Group. Group administrators provide information about Group Schedules to the system of the present invention.

Practice Schedules are the general working hours for Providers that are not a member of a Group. Vacation Schedules are scheduled time off from regular working hours. Information about Practice and Vacation Schedules are supplied by the Providers themselves, a Facility, or a Group administrator.

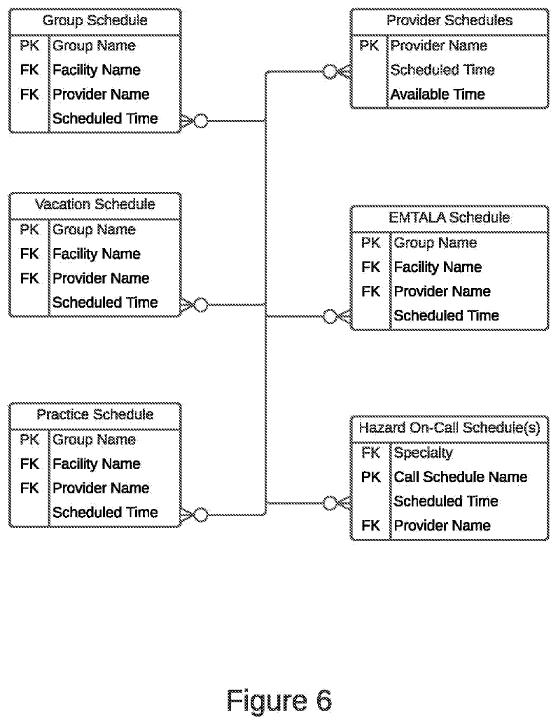

FIG. 6 shows a database table relationship diagram showing the connection between various Provider schedules. Information about all applicable Schedules (Group, Vacation, Practice, EMTALA, Hazard On-Call), may be viewed at once, under a Provider Schedules table. This allows the Administrator to see when Providers are available, to quickly create a new Hazard On-Call Schedule.

The ability to link Providers' schedules is a fundamental feature of the present invention. This is superior to the existing ESAR-VHP call list, in that the present invention provides information on the current and future availability of Providers. Currently, when using ESAR-VHP, an Administrator is simply given biographical and contact information for each Provider. They are constrained to contacting emergency response Providers without any knowledge of their availability.

### Administration of Scheduling and Credentialing

Administrators manage the scheduling and credentialing for Facilities. In a Hazard Event, the Administrator will also manage the creation of a Hazard On-Call Schedule. Three categories of Hazard On-Call Schedules are available, those responding to a specific Hazard Event, those responding to an anticipated Hazard Event, and those responding to All-Hazard Events.

When responding to a Hazard Event, an Administrator may create a specific new Hazard On-Call Schedule to organize Providers. For example, if the Hazard Event was an oceanside building collapse, an Administrator at a nearby Facility may create a new schedule called the Oceanside Collapse On-Call Schedule and start filling the schedule with appropriate emergency physicians as well as construction crews and ambulance drivers, among others. In this embodiment of the invention, an Administrator creates a new Hazard On-Call Schedules as the need arises.

In another embodiment of the invention, an Administrator may create a particular Hazard On-Call Schedule prior to a Hazard Event. For example, during the summer months in a dry climate, the Administrator may build a new schedule called a Heat Relief Hazard On-Call Schedule. If a heat wave occurs, the Administrator will then activate the schedule and Providers will be on-call. In some embodiments, the Providers are added to the Heat Relief Hazard On-Call Schedule prior to activation. The Administrator may add and remove Providers as needed during the Hazard Event.

Another embodiment of the invention utilizes a Hazard On-Call Schedule for an All-Hazard Event. This schedule may include a variety of Providers, who are prepared for any scenario and capable of responding to most hazards. The All-Hazard Event schedule may be created in advance of the Hazard Event.

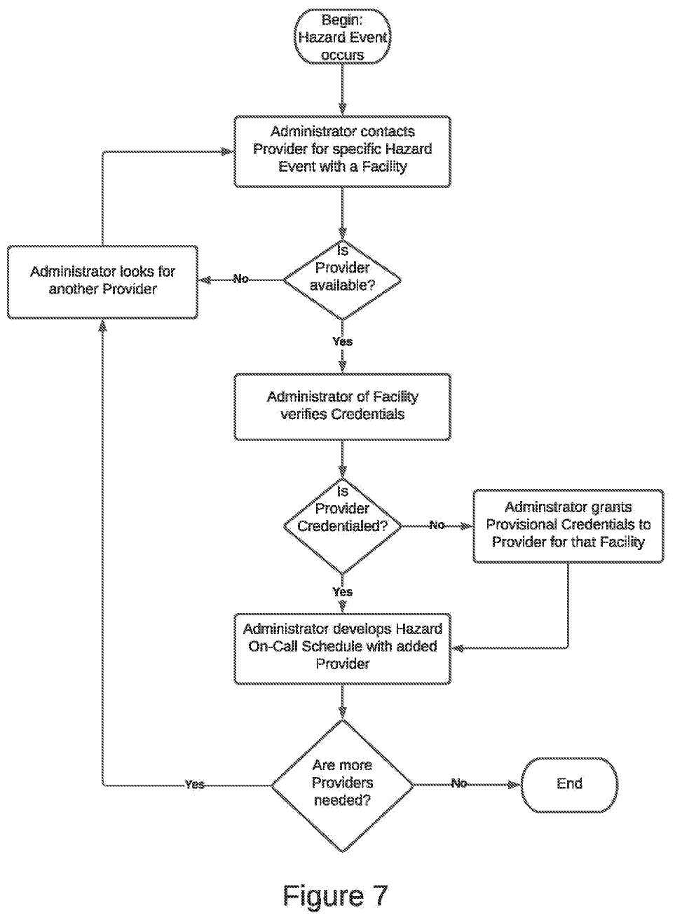

FIG. 7 is a flowchart demonstrating one embodiment of the method for creating and adding Providers to a Hazard On-Call Schedule. This scenario applies to the creation of a Hazard On-Call Schedule for a specific Hazard Event.

When a Hazard Event occurs, the Administrator at a nearby Facility begins searching for Providers with a specific set of skills appropriate for responding to the Hazard Event. The Administrator views a list of Providers enrolled in the system, similar to the Providers list shown in FIG. 3, and may find more information by looking at individual Providers, as shown in FIG. 4. Administrators may search for the Providers based on proximity to the Hazard Event, skills, State Medical License, schedule, or any other data in the system.

As the Administrator finds Providers, they contact them to see if the Provider is available and interested in responding to the Hazard Event. In alternative embodiments, the Provider has already preauthorized themselves for service in a Hazard On-Call Schedule and waives the need for initial contact from the Administrator. These Providers are automatically added to the Hazard On-Call Schedule.

If a Provider is available and interested in serving, the Administrator then verifies the Provider's Credentials. If the Provider is already credentialed at the Facility, they can be added to a new Hazard On-Call Schedule. If the Provider is not credentialed, the Administrator may grant Credentials at that time.

When granting Credentials, the Administrator may review pertinent information about the Provider in the system. The Administrator is in the best position to make judgments on who can serve at the Facility, on a regular basis as well as in an emergency. The Administrator may grant Provisional Credentials to the Provider, allowing them to serve under the Facility for a limited time, and for a specific set of privileges (medical procedures) depending on their capabilities and licenses. Depending on the situation, and the local and hospital regulations, an Administrator may grant Provisional Credentials to out-of-state Providers if necessary. If Provisional Credentials are granted, the Provider Table is updated to show the new Provisional Credentials, displaying any pertinent limitations including the expiration date, location, and authorized services.

This rapid credentialing process is an advantage over the current means of credentialing. The normal credentialing process is typically extensive and comprehensive. Usually, a Provider will apply for Credentials by submitting ten to fifty pages of information about themselves to a Facility. In the present invention, the relative pertinent information about the Provider is already in the system, and the Administrator can make an informed decision on Provisional Credentials immediately.

Once the Provider's Credentials or Provisional Credentials are verified, the Administrator may add them to a Hazard On-Call Schedule responding to the Hazard Event. When scheduling the Provider, the Administrator will take the Provider's current EMTALA, Group, Practice, and Vacation Schedules into consideration. In some circumstances, and at some Facilities, the Administrator may disregard existing schedules, and deem the current Hazard On-Call Schedule more important than the other schedules. In other circumstances, the Administrator may prioritize the existing EMTALA Schedule, and/or other schedules. The Administrator is in the best position to make these decisions and has authority to do so for Providers at their respective Facilities.

Once a Provider is added to the Hazard On-Call Schedule, the Administrator determines whether more Providers are needed, and repeats the process. Over the course of the Hazard Event response, the Administrator may add or remove Providers from the Hazard On-Call Schedule as needed.

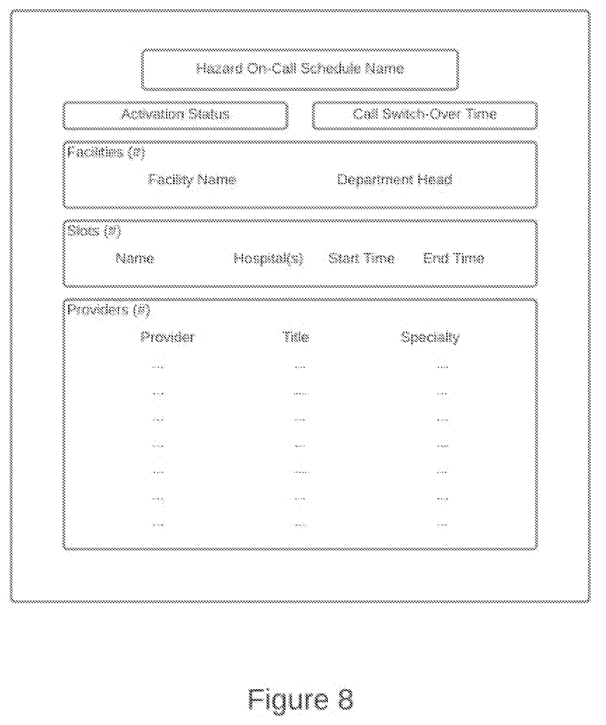

FIG. 8 is an example display of a Hazard On-Call Schedule. All Providers and their respective on-call times are shown in one location. As the Providers are added, or removed, from a Hazard On-Call Schedule, their scheduled time is updated in the Provider Schedule shown in FIG. 6. It is important to always maintain all the various schedules to ensure knowledge of when a Provider is available, as more than one Administrator may seek to schedule the same Provider at the same time for more than one Hazard On-Call Schedule.

Once a Hazard On-Call Schedule is established, the Administrator may call Providers as needed, and know that they will respond and have appropriate Credentials to serve. As the Hazard Event subsides, and emergency response Providers are no longer needed, the Administrator may terminate the Hazard On-Call Schedule and update the Providers' schedules in kind.

The web application exchanges information with Administrators through a communications interface with Administrators' devices. Administrators' devices are comprised of a memory, processor, communications interface, an input device, and an output display. A typical Provider device might be a personal computer, smartphone, or tablet.

### Communications

An essential part of the invention is the ability to communication along several pathways. These communication pathways include the ability to contact all Users of the entire system, all Users at a single Facility, all Users in a Group, and all Providers. Other arrangements of Users for communication are also envisioned.

The contact information presented in the Provider Table affords ease of communication. One embodiment of the invention includes the ability to send Hazard Event alerts to Providers. These alerts may utilize any number of means (e.g., SMS, email, voicemail, online messenger), and target a specific region, specialty, Group, Facility, or all Providers. Alerting Providers of a Hazard Event may prompt them to reach out to Administrators, proactively accelerating the process of filling a Hazard On-Call Schedule.

In addition to urgent messaging, the invention provides communications for training and event preparedness. Training messages may include guidance on how to use the invention's system, as well as information on best practices and emergency preparedness for related or general Hazard Events.

In some embodiments, the Windows Service controls system communications. Regularly scheduled, or automatically prompted, messages and reports may be sent to Providers and Administrators. One embodiment sends emails to Providers ever Monday morning, alerting them to their respective Hazard On-Call Schedules, with a link to the pertinent web application schedule display and an attached spreadsheet for downloading.

### Examples

In one application of the invention, a hospital Administrator in a large city may use the system to respond to a pandemic. The Administrator may need additional registered nurses, primary care physicians, emergency room physicians, and intensive care unit physicians. The Administrator may search the Provider Table for preferred specialties and nearby Providers. The Administrator may provide Provisional Credentials as needed and immediately add the Providers to the Hazard On-Call Schedule, satisfying the imminent need and planning for continued heightened needs.

In another application, the Federal Emergency Management Agency (“FEMA”) may set up a field hospital in a rural area in response to a hurricane. An official with access to the invention may require several different types of personnel to supplement the field hospital. Examples might include emergency care physicians and other medical personnel, but also search and rescue teams, and temporary shelter construction and management. The official will have access to a list of Providers in the surrounding area and may give Provisional Credentials to serve at the FEMA Facility.

The system of connecting and organizing information on the specialty, availability and location of Providers, and the method of selecting, credentialing, and scheduling the Providers can apply to numerous situations and locales. It is envisioned that local hospitals, city and state agencies, federal agencies and non-profit organizations may all find the invention useful in responding to anticipated and unanticipated Hazard Events of all kinds.

While there have been shown and described illustrative examples of the comprehensive system and method for scheduling and credentialing emergency response personnel, it is to be understood that various other adaptations and modifications may be made within the spirit and scope of the embodiments herein. Thus, while the foregoing description has been directed to specific embodiments, it will be apparent that other variations and modifications may be made to the described embodiments, with the attainment of some or all their advantages. Accordingly, this description is to be taken only by way of example and not to otherwise limit the scope of the embodiments herein.

We claim:

1. A system to respond to a Hazard Event by creating a Hazard On-Call Schedule of Providers, comprising:
at least one external data source with a communication interface to the Internet, wherein the at least one external data source is comprised of data regarding the Credentials and the schedules of at least one Provider;
the at least one Provider has at least one provider electronic device that includes a processor, memory, a communication interface to the Internet, at least one input device, and at least one output display;
at least one Facility has at least one facility electronic device that includes a processor, memory, a communication interface to the Internet, at least one input device, and at least one output display;
a web application hosted on a web server that includes a processor, memory, and a communication interface to the Internet, which is connected to the at least one external data source, the at least one provider electronic device, and the at least one facility electronic device; and
wherein the web application presents the data regarding the Credentials and the schedules of the at least one Provider to the at least one Administrator at the at least one Facility, to facilitate the verification of Credentials, assessment of availability based on the schedules, and to add the at least one Provider to the Hazard On-Call Schedule.

2. The system of claim 1, wherein the Credentials of the at least one Provider are updated to reflect authority to serve at the at least one Facility.

3. The system of claim 2, wherein the Credentials are limited by time, location, and privileges.

4. The system of claim 1, wherein the at least one data source is a database managed by one of the following entities. the at least one Facility, the Centers for Medicare and Medicaid Services, a state or territory medical board, the Drug Enforcement Administration, the at least one Provider.

5. The system of claim 4, wherein the at least one data source's data about the at least one Provider is further comprised of one of the following State Medical License, NPI, DEA License, fellowship status.

6. The system of claim 4, wherein the schedules of the at least one Provider include one of the following: EMTALA On-Call Schedule, Group Schedule, Practice Schedule, Vacation Schedule.

7. The system of claim 4, wherein the web application presents data regarding the at least one Provider limited by one of the following: location, State Medical License, DEA License, specialty.

8. The system of claim 1, wherein the web application presents availability data regarding the at least one Provider limited by one of the following: time, location, Facility, type of hazard, type of medical procedure.

9. A method to respond to a Hazard Event by creating a Hazard On-Call Schedule of Providers, comprising:
providing at least one external data source with a communication interface to the Internet, wherein the at least one external data source is comprised of data regarding the Credentials and the schedules of at least one Provider;
providing the at least one Provider has at least one provider electronic device that includes a processor, memory, a communication interface to the Internet, at least one input device, and at least one output display;
providing at least one Facility has at least one facility electronic device that includes a processor, memory, a communication interface to the Internet, at least one input device, and at least one output display;
providing a web application hosted on a web server that includes a processor, memory, and a communication interface to the Internet, which is connected to the at least one external data source, the at least one provider electronic device, and the at least one facility electronic device,
transmitting the data regarding the Credentials and the schedules of the at least one Provider to at least one Administrator at the at least one Facility;
the at least one Administrator verifying the Credentials of the at least one Provider;
the at least one Administrator assessing the availability of the at least one Provider based on the schedules of the at least one Provider; and
the at least one Administrator adding the at least one Provider to the Hazard On-Call Schedule.

10. The method of claim 9, wherein the at least one Administrator determines the at least one Provider lacks Credentials at the at least one Facility and grants the at least one Provider Provisional Credentials to serve at the at least one Facility.

11. The method of claim 10, wherein the Provisional Credentials are limited by time, location, and privileges.

12. The method of claim 9, wherein the at least one data source is a database managed by one of the following entities: the at least one Facility, the Centers for Medicare and Medicaid Services, a state or territory medical board, the Drug Enforcement Administration, the at least one Provider.

13. The method of claim 12, wherein the at least one data source's data about the at least one Provider is further comprised of one of the following: State Medical License, NPI, DEA License, fellowship status.

14. The method of claim 13, wherein the schedules of the at least one Provider include the at least one Provider's EMTALA On-Call Schedule.

15. The method of claim 14, wherein the at least one Administrator adds the at least one Provider to the Hazard On-Call Schedule during time periods that do not conflict with the EMTALA On-Call Schedule.

16. The method of claim 13, wherein the schedules of the at least one Provider include the at least one Provider's Group Schedule.

17. The method of claim 13, wherein the schedules of the at least one Provider include the at least one Provider's Practice Schedule.

18. The method of claim 13, wherein the schedules of the at least one Provider include the at least one Provider's Vacation Schedule.

19. The method of claim 13, wherein the at least one Administrator searches for the at least one Provider by one of the following limiting criteria: location, State Medical License, DEA License, specialty.

20. The method of claim 9, wherein the at least one Provider limits availability based on one of the following time, location, Facility, type of hazard, type of medical procedure.
